(19) United States
(12) Patent Application Publication
(10) Pub. No.: US 2011/0172491 A1
(43) Pub. Date: Jul. 14, 2011

(54) DETACHABLE BALLOON CATHETER

(75) Inventors: Gregory Piskun, Moranville, NJ (US); Dan Rottenberg, Haifa (IL); Boaz Manash, Givat-Ada (IL); Dima Pinhasov, Naharia (IL)

(73) Assignee: Macroplata, Inc., Morganville, NJ (US)

(21) Appl. No.: 12/895,313

(22) Filed: Sep. 30, 2010

Related U.S. Application Data
(60) Provisional application No. 61/247,605, filed on Oct. 1, 2009.

(51) Int. Cl. A61B 1/018

(52) U.S. Cl. 600/104

(57) ABSTRACT

The disclosure relates to a device and method such as for treatment of a gastrointestinal perforation and a gastrointestinal bleeding. The device can be a balloon catheter that controls bleeding by pressing on a bleeding area or/and prevents the gastrointestinal contents trespassing outside a gastrointestinal lumen into a body cavity by blocking an opening in the luminal wall or blocking the colon distal or proximal to the perforation. The preferred device is inserted using an endoscope and allows to partially or completely withdraw an endoscope while leaving the balloon at the target area. The device and method can facilitate ceasing a colonic bleeding and blocking colonic perforation.

### FIELD

The present disclosure relates to catheter assemblies, such as balloon catheters.

### CROSS REFERENCE TO RELATED APPLICATION

This application claims benefit of U.S. provisional patent application No. 61/247,605, filed Oct. 1, 2009, the disclosures of which is incorporated by reference in its entirety herein.

### BACKGROUND

Gastrointestinal bleeding is a common and potentially life-threatening medical condition. Colon bleeding, for instance, is frequently encountered during polypectomy (polyps removal), and excision of colonic tumors. Gastrointestinal bleeding can typically be treated by applying a local pressure to a bleeding area, for example, by temporary manual or instrumental compression or by compression using staplers, clips, magnets, or/and rubber bands. Alternatively, a variety of energy sources, including but not limited to electrical, ultrasonic, thermal coagulation, can be used to treat the bleeding.

A colonic perforation can occur by applying excessive mechanical force or excessive energy to a colonic wall. A colon perforation is a life-threatening surgical emergency, requiring an expeditious surgical closure of a colonic perforation to preclude fecal contamination of an abdominal cavity and resulting sepsis. A bleeding tamponade using balloon is a known technique (for example, balloon tamponade of the esophageal varices) of stopping a local bleeding. Such balloons are inflated for a short period of time in order to compress a bleeding vessel, until natural or therapeutic coagulation takes place.

Several types of balloon catheters are known to facilitate movements of an endoscope inside a gastrointestinal tract. Publication No. WO2007017854 describes a balloon-guided endoscopic system using two balloons, where one balloon is placed on the external surface an endoscope, and the other balloon is carried by the catheter, which is inserted into one of the channels of an endoscope. The balloons are sequentially inflated, while an endoscope is pushed along the bowel for the segmental bowel inspection between the balloons. Also, Patent Publication Nos. JP20060088928, US2004/0933693, and IE20020000823 describe balloon catheters that facilitate the endoscope's advancement along the intestinal tract.

A diameter of a working channel of a modern standard colonoscope is typically very small, being approximately between 2.8 to 3.7 mm, which limits the diameter and, hence, functionality of devices like balloon catheters that are inserted in such channels. Currently, there are very small diameter-balloon catheters on the market which can be inserted into an endoscope's working channel. These catheters, however, have significant limitations precluding their effective use for a bleeding control. Current balloon catheters have a proximal Luer connector, accommodating standard syringes. Industry-standard Luer connector metrics, according to ISO 594/1 standards, provide a Luer with a conical fitting of a 4.3 mm diameter and external Luer diameter of about 7.0 mm. As a result, when a standard balloon catheter is inserted into a working channel of an endoscope and is inflated, a Luer connector precludes a withdrawal of an endoscope from a patient while leaving the balloon inside a colon.

### SUMMARY

The present disclosure relates to a catheter assembly that includes a detachable connector member, which can enable an endoscope to be withdrawn from the catheter without requiring the removal of the catheter from the patient. In a preferred embodiment, the catheter assembly includes a catheter shaft defining an interior channel and having an outer cross-sectional size selected to be received through a working opening of an endoscope. The preferred embodiment also has a valve associated with the catheter shaft in fluid connection with the interior channel for opening and closing the interior channel. The valve can be configured for fluid connection to a connector member for passing a fluid between the connector member and the interior channel when the valve is open, wherein the valve has an outer cross-section configured and dimensioned for reception through the working opening when detached from the connector member. The preferred catheter is a balloon catheter, and more preferably a gastrointestinal balloon catheter, that includes a balloon in fluid connection with the interior channel for inflation and deflation by passing the fluid through the channel. The preferred endoscope is a colonoscope, and the preferred connector member is a Luer connector.

The valve and connector member can be configured to cause the valve to open automatically based on a connection between the valve and connector member, and to cause the valve to close automatically upon disconnection thereof. The valve can be configured to open, for example, upon the connection of the valve and connector member or, as another example, upon the connection of a syringe with the connector member when the connector member is connected to the valve.

The connector member can include a valve opening member configured to displace a portion of the valve to open the valve. Also, the valve can include a valving obstruction, such as a ball in embodiments in which the valve is a ball valve. The valving obstruction preferably has a closed position, blocking the flow through the valve, to close the valve. The valve opening member is configured for engaging and moving the valving obstruction to an open position, unblocking the flow through the valve, to open the valve when the connection member and valve are connected. The connector member can define an interior connector channel having the valve opening member, which can extend from the connector channel into the valve and move the valve obstruction to the open position. A biasing member can be provided to resiliently bias the valving obstruction towards the closed position. The valve opening member can be slidably received and disposed in the connector channel so that the connecting of the connector member to a syringe causes the syringe to displace the valve opening member against the valving member and open the valve. In another embodiment, the valve includes a seal and the connector channel includes a piercing member fluidly connected with the connector channel and configured to pierce the seal to open fluid communication between the connector channel and the interior channel of the shaft when the connector member and valve are connected.

The shaft can have an outer shaft diameter at a distal end portion thereof, and the valve can be connected to the distal end portion and have an outer valve diameter substantially the same or smaller than the shaft diameter. In some embodiments, the valve diameter is no larger than, or is smaller than, the shaft diameter.

Preferably, the connector member includes a fitting configured for engaging the valve in a releasable snap connection therewith.

In a preferred method for an endoluminal treatment, a balloon catheter is connected to a connector member. A balloon catheter is inserted through a working opening of an endoscope and is positioned endoluminally, and a balloon of a balloon catheter is inflated. The inflated balloon catheter is detached from a connector member, thereby closing a valve associated with the catheter to block fluid flow from the catheter. The endoscope can then be removed from the catheter, such as by sliding the valve through the endoscope's working opening.

The balloon can be inflated to apply pressure against a bleeding site to stop or slow the bleeding. Alternatively, the balloon can be positioned to block a perforated region in a wall of the lumen. In one embodiment, the balloon catheter is inflated using a syringe connected to the Luer connector, and a syringe is connected or can be reconnected to the Luer connector after removal of the endoscope for deflating or re-inflating the balloon.

### BRIEF DESCRIPTION OF THE DRAWINGS

The drawing figures depict one or more implementations in accordance with the present concepts, by way of example only, not by way of limitations. In the figures, like reference numerals refer to the same or similar elements.

FIG. 1 is a perspective view of an embodiment of the balloon catheter inside an endoscope's working channel;

FIG. 2 is a schematic view of a distal portion of the balloon catheter of FIG. 1 disposed inside a perforated colon;

FIG. 3 is a schematic view thereof blocking the colon distal or proximal to the perforation;

FIG. 4a is a cross-sectional view of a valve connector of the catheter of the embodiment of FIG. 1;

FIG. 4b is a cross-sectional view thereof connected to a Luer of the embodiment of FIG. 1;

FIG. 5 is a perspective view of a connection and valve assembly of another embodiment having a rubber seal valve;

FIGS. 6a and 6b are cross-sectional views of a Luer and valve assembly of the embodiment of FIG. 5, respectively before and after snap connection;

FIGS. 7a and 7b are perspective views of a distal end of an embodiment of a balloon catheter, respectively with the balloon deflated and inflated; and

FIGS. 8a and 8b illustrate steps in an embodiment of the disclosure.

### DETAILED DESCRIPTION

The preferred embodiment of the present disclosure is directed to balloon catheter, such as configured for treatment of a gastrointestinal perforation and a gastrointestinal bleeding or for use in another body lumen. For certain procedures, the device can include a balloon catheter that controls bleeding by pressing on a bleeding area or/and prevents the gastrointestinal contents trespassing outside a gastrointestinal lumen into a body cavity by blocking an opening in the luminal wall or blocking the colon distal or proximal to the perforation. The device is preferably inserted using an endoscope in a manner that allows partial or complete withdrawal of the endoscope while leaving the balloon at the target area. The preferred device and method facilitate ceasing a colonic bleeding and/or blocking colonic perforation.

The balloon catheter device of the preferred embodiment, which is preferably configured to enable leaving a deployed balloon catheter inside a colon at the target area while removing an endoscope, will facilitate the treatment of a gastrointestinal bleeding and/or gastrointestinal perforation. This also can facilitate a cost-effective management of the medical facility's resources. Therefore, there is a need for such a device.

The balloon catheter preferably includes a removable Luer and a miniature valve mechanism, both located at the device's proximal end. The removable Luer, when attached to the proximal end of the balloon catheter, engages and opens the miniature valve. Consequently, a standard syringe attached to the Luer can now be used for balloon inflation. When the Luer is removed, the valve becomes disengaged and automatically closes, hence, preventing air or other fluid from exiting the balloon. An endoscope can now be removed without deflating the balloon. The balloon, therefore, remains inflated, although the Luer and an endoscope are now removed.

The external diameter of a miniature valve is preferably the same or smaller than the diameter of an endoscope's working channel. This permits a retraction of an endoscope from the patient, leaving the inflated balloon inside the desired location in the patient's colon. The balloon is preferably made of a compliant material allowing inflating the balloon to a relatively large diameter. The balloon can have a variety of shapes, including but not limited to, a substantially round or substantially elliptical or substantially irregular shape. The diameter of an inflated balloon is larger than the diameters of a catheter's shaft and/or the balloon in folded or deflated configurations.

In a preferred embodiment, the technology and method enhance a physician's ability to treat a colon bleeding and/or perforation. Such method can include:
1) Inserting the non-deployed balloon catheter (deflated) through the endoscope into the patient's lumen,
2) Positioning the balloon adjacent to the bleeding location,
3) Inflating the balloon,
4) Continuing inflating the balloon until a sufficient pressure from the balloon is applied to the bleeding site and bleeding is stopped,
5) Removing the removable Luer connector from the proximal end of the balloon catheter,
6) Removing (partially or completely) the endoscope,
7) Leaving the balloon catheter in place for a period of time selected by a physician until bleeding is stopped (physician may intermittently inflate and deflate balloon as clinically indicated) or, if bleeding is not stopped by the balloon, until a patient is transferred to a Surgical Suite for an operation,
8) Clinically observing and hemodynamically stabilizing the patient in a monitoring medical facility until bleeding is ceased or patient is transferred to a surgical suite.
9) If bleeding is controlled, re-connecting the Luer connector, deflating the balloon and removing the balloon catheter.

A preferred method for colonic perforation control includes:
1) Inserting the non-deployed balloon catheter (deflated) through the endoscope into the lumen,
2) Positioning the balloon adjacent to the site of the perforation,

If perforation is relatively small (less than 2-3 cm in diameter):
3) Inflating the balloon against the perforation,
4) Continue inflating the balloon until it completely blocks the perforation and, hence, isolates the abdominal cavity from colonic lumen,

If perforation is relatively large (larger that 2-3 cm in diameter):
3) Inflating the balloon distal or proximal to the perforation,
4) Continuing to inflate the balloon until it completely occludes a distal or proximal to a perforation colon, and, hence, minimizes contamination of the abdominal cavity with colonic contents,

When balloon is inflated to a desired degree:
5) Removing the removable Luer connector from the proximal end of the balloon catheter,
6) Removing the endoscope,
7) Leaving the balloon catheter in place and prepare patient for an endoluminal or/and extra-luminal repair of a colonic perforation, including if necessary transferring a patient to a Surgical Suite for a conventional or laparoscopic operation,
8) Clinically observing and hemodynamically stabilizing the patient in a monitoring medical facility until a surgeon and surgical suite are prepared for an operation, hence, further minimizing an intra-abdominal fecal contamination.

In the various procedures, the balloon can remain disposed endoluminally during the surgical operation to function as a “marker” of a perforation site, and, hence, guide a surgeon in locating the perforation. In addition, an inflated balloon can function as an endoluminal tissue retractor, which facilitates a surgical closure of a colonic wound.

During the period of a patient's observation, an endoscope is removed, cleaned and used for another patient. This method, therefore, allows for a cost-effective approach to utilization of health care resources, including endoscopic equipment, facility, and medical personnel.

In addition to the described colonic applications, the device and methods can be used inside other body lumens during endoscopic procedures. Other methods of placing the balloon endoluminally, such as to minimize a patient's blood loss, can be used, which can eliminate or reduce the need for blood transfusion, for example, during preoperative management of a bleeding patient.

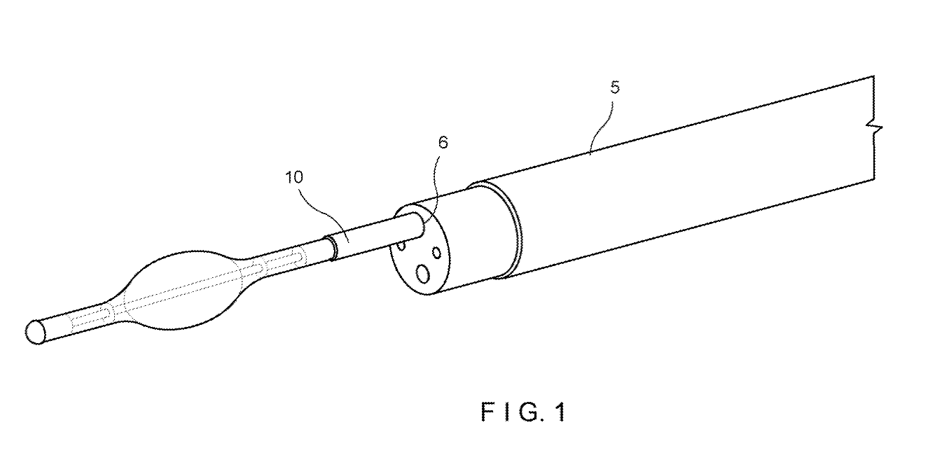

Referring to FIGS. 1-3, the preferred embodiment of the disclosure includes a balloon catheter 10 configured to control bleeding by pressing on a bleeding area. In one embodiment, the catheter is configured as a gastrointestinal balloon catheter for preventing the gastrointestinal contents trespassing outside a gastrointestinal lumen into a body cavity by blocking an opening 3 in the luminal wall 4. For example, the device can be used for treatment of a colon 1 perforation 2 and colon bleeding 3. In a preferred method of this treatment, the catheter 10 is inserted through a working channel 6 of an endoscope, such as the standard endoscope 5 shown in FIG. 1, which can be a colonoscope. After balloon 11 is inflated, the endoscope 5 is withdrawn from the patient, leaving an inflated balloon 11 at a desired location.

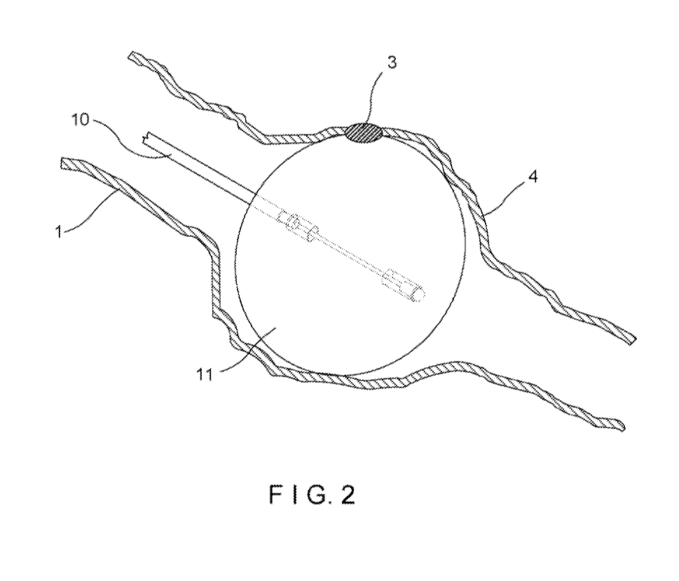

As shown in FIG. 2, if bleeding, for example bleeding into the colon, needs to be stopped during an endoscopic procedure, the treatment can be done by (1) inserting the deflated balloon catheter 10 through the working channel 6 of the endoscope 5; (2) positioning the balloon 11 of the balloon catheter 10 adjacent to, and preferably in abutment against and covering the bleeding site 3; (3) inflating the balloon 11 to stop the bleeding; (4) removing Luer connector 20 assembly from the balloon catheter 10; (5) removing the endoscope 5 from the colon while leaving the balloon catheter 10 in place for some time until bleeding is stopped; (7) re-connecting the Luer connector; (8) deflating the balloon; and (9) removing the balloon catheter 10 from the colon.

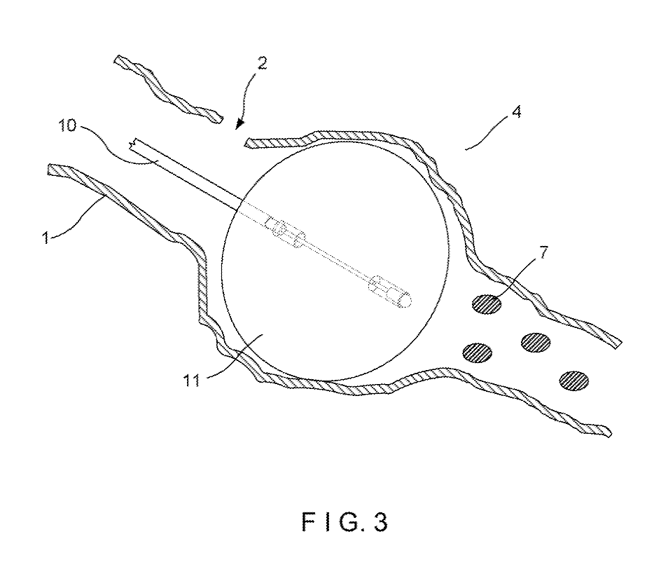

As shown in FIG. 3, a treatment of a colon perforation can be done as described above for treating the bleeding site, but the balloon 11 is positioned adjacent to or against the perforation 2 if the perforation 2 is relatively small, distal or upstream with respect to the perforation 2 if perforation 2 is relatively large. The balloon 11, therefore, blocks the perforation from the upstream portion of the colon or intestinal tract and prevents or decreases a fecal contamination 7 of the body cavity. The blockage of a perforation by the balloon as described permits additional time for a patient's stabilization and better preparation for surgery.

The device and method of the preferred embodiments allow disconnecting a patient from the endoscopic equipment and transferring a patient from an endoscopic unit, with the balloon deployed endoluminally, to an intensive care unit for close monitoring and preparation to surgery. With some embodiments of the present device and method, the inflated balloon catheter can be left at the site of the bleeding or perforation for several hours when clinically indicated. In the meantime, the endoscopic equipment can be cleaned and used for another patient, hence, facilitating a cost-effective use of expensive capital equipment.

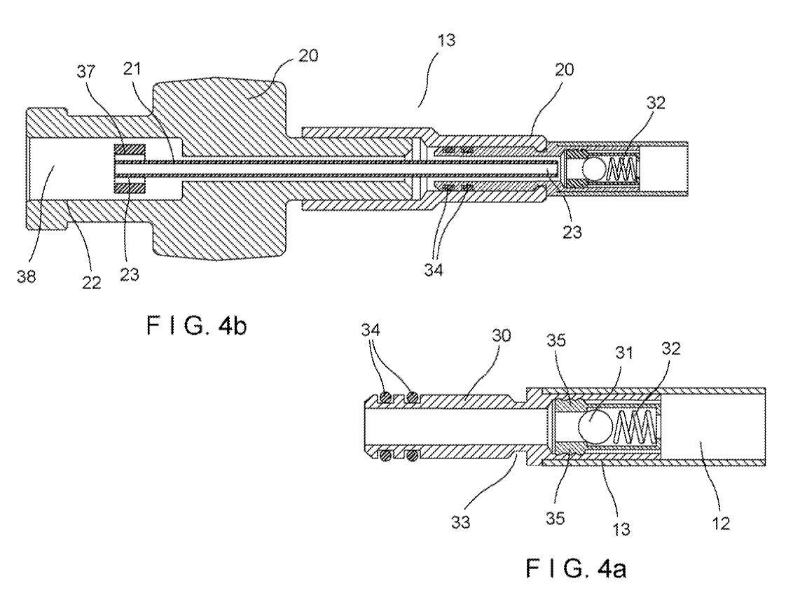

As shown in FIGS. 4a and 4b, an embodiment of the device includes a balloon catheter 10, which includes a removable Luer assembly 20 and a valve assembly 30, both located at the catheter device's proximal end 13. The valve assembly 30 is configured and dimensioned to fit through the working channel 6 of the endoscope 5, which typically has an inside diameter of between 2.8 and 3.7 mm. The outside of the valve assembly 30 is preferably the substantially the same axial cross-sectional size or diameter, and more preferably is no larger, or is smaller, in axial cross-section or diameter than the shaft 12, or working channel, of the catheter 10 to which it is connected, permitting the valve assembly 30 and a portion of the shaft 12 through the working channel 6. The outside embodiment of the valve assembly 20 in some embodiments is between about 2 mm and 4 mm, and typically less than about 3 mm. Other suitable diameters can be used depending on the configuration of the endoscope, such as up to about 10 mm in diameter.

The removable Luer assembly 20 can be configured to industry standards, such as according to ISO 594/1 standards—having an about 4.3 mm diameter conical fitting and external Luer diameter of about 7.0 mm, although other sizes can be used to connect to a syringe or other inflation fluid pumping device. When the Luer assembly 20 is attached to the proximal end of the balloon catheter and associated with the valve assembly 30 thereof, engages and opens the miniature valve assembly 30, and allows a standard syringe to be attached to the Luer assembly 20 and used for balloon inflation or deflation. When the Luer assembly 20 is removed from the valve assembly 30, the valve assembly becomes disengaged and automatically closes, preventing balloon inflation fluid, such as air or a liquid, from exiting the shaft 12 and balloon 11. The endoscope 5 can at this point be removed from the catheter 10 without deflating the balloon. The balloon 11, therefore, remains inflated, although the Luer and an endoscope are now removed from the catheter 10.

As indicated above, the external diameter of a miniature valve assembly is preferably smaller than the internal diameter of an endoscope's 5 working channel 6. This permits the retraction of the endoscope 5 from the patient, leaving the inflated balloon inside the desired location in the patient's colon.

When the Luer assembly 20 is attached to the valve assembly 30, in one embodiment any standard syringe can be used to inflate or deflate balloon 11. When the Luer assembly 20 is removed from the valve assembly 30, valve 31 in valve assembly 30 is automatically closed, preventing inflation fluid from exiting, keeping balloon 11 inflated. Because the diameter of a miniature valve assembly 30 is less than the diameter of the working channel 6 of the endoscope 5, the endoscope 5 can be retracted from the patient, leaving the inflated balloon 11 inside the desired location in the patient's colon.

In the embodiment of FIGS. 4a and 4b, miniature valve assembly 30, which is preferably attached to the shaft 12 of a proximal end 13 of balloon catheter 10, is configured as a ball valve and includes a small ball 31 or other valving obstruction, received within channel 36 of the valve assembly, which is in fluid communication with the interior of the shaft 12 of the catheter 10. The valve assembly 30 also includes a biasing member, such as spring 32. The spring 32 preferably resiliently biases the ball 31 into abutment against valve seat 35 to block inflation fluid from exiting the balloon 11 when the Luer assembly 20 and its syringe bar 21 are disengaged therefrom. Alternative embodiments can use other types of valves.

Luer assembly 20 includes Luer connection 22 configured for attaching to a corresponding Luer connector from a syringe, although alternative embodiments can include a connector member that is integral with a syringe or other fluid-transfer or pumping device. A valve opening member, such as a syringe bar 21, is provided in the Luer assembly 20 to open the valve 30 when the Luer 20 and valve assemblies 30 are connected. The preferred syringe bar 21 defines holes and a channel 23 at its proximal and distal sides to receive and deliver the inflation fluid from or to a syringe to or from the valve assembly 30. The syringe bar 21 preferably is preferably axially elongate and configured and associated with the Luer assembly body for extending into the housing of the valve assembly to displace the ball 31 from the valve seat 35. Most preferably, the syringe bar 21 is axially slidably received within in the Luer assembly 20 and configured for being displaced axially by the tip of a syringe that is attached to the Luer assembly 20 when the Luer and valve assemblies 20,300 are connected. An abutment member 37 can be provided on a proximal side of the syringe bar to provide a wider surface area to abut against a syringe tip so that the syringe can displace the syringe bar 21 forward to displace ball 31 and open the valve. The syringe bar 21 can be disposed within the fluid channel 38 of the Luer assembly. A distal side of the Luer assembly 20 includes a valve attachment, which in one embodiment includes snap arms 24, although other attachments can be employed to secure the Luer assembly 20 to the valve assembly 30.

When Luer assembly 20 is pushed against valve assembly 30, snap arms 24 engage groove 33 or other suitable feature, such as on the outside of the valve assembly 30, and a seal, such as O-rings 34, provides a fluid seal between the two assemblies. The snap arms 24 preferably provide a disengageable snap connection between the valve and Luer assemblies 30,20, although threaded or other connectors can be employed in other embodiment. If a syringe is pushed into Luer connection 22, it displaces bar 21, which in turn pushes ball 31 against spring 32, to allow the filling fluid to flow to and from balloon 11.

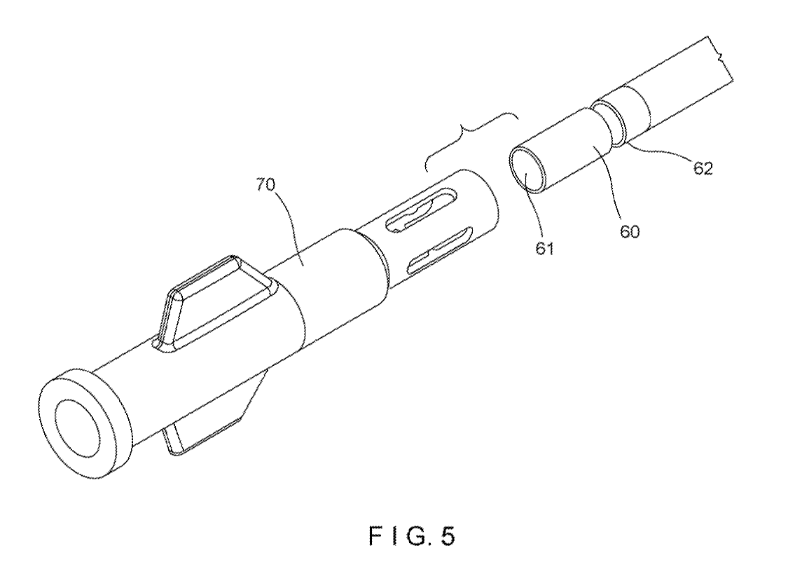

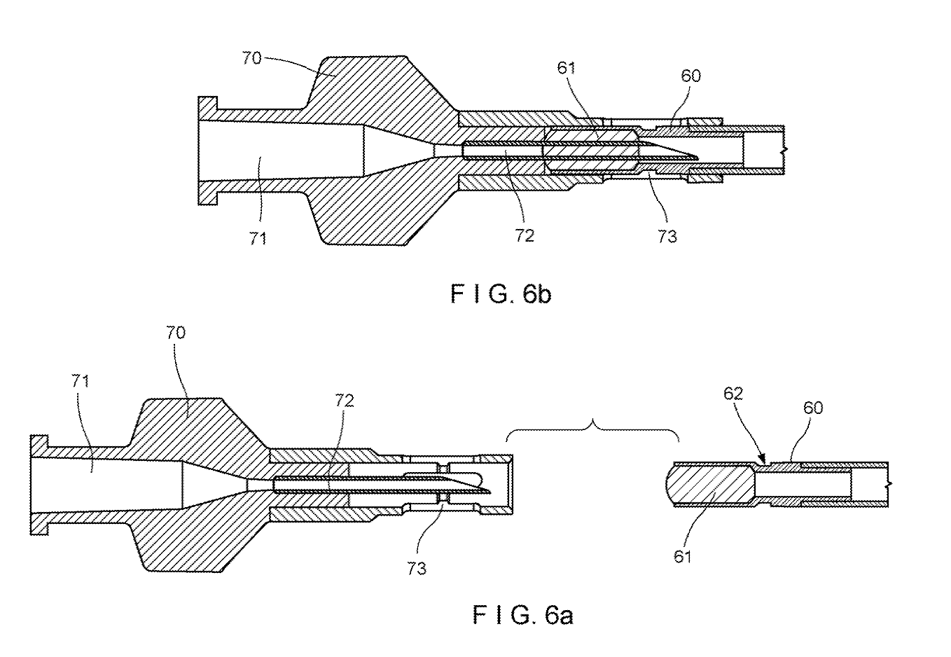

Referring to FIGS. 5, 6a, and 6b, in another embodiment, miniature valve assembly 60 includes a pierceable seal 61, such as of an elastomer, including rubber, silicon, or soft polyurethane seal 61 and groove 62. Luer assembly 70 includes Luer connection 71 for a syringe attachment, a piercing member such as short needle 72, and snap arms 73. When Luer assembly 70 is pushed against valve assembly 60, snap arms 73 engage groove 62 in valve assembly 60, and needle 72 punctures seal 61, opening a fluid communication to balloon 11. When Luer assembly 70 is pulled from valve assembly 60, the snap arms 73 are released from groove 62, and needle 72 is retrieved therefrom. The seal 61 is preferably configured to reseal itself upon withdrawal of the needle 72, closing the fluid communication to balloon 11. In an alternative embodiment, the needle can be mounted slidably within the channel of the Luer assembly 20 to be pushed forward to pierce the seal when a syringe is attached to the Luer assembly 20.

Other types of valves can by employed in other embodiments, including a self closing, leaflet valves (such as bi, tri, and quad leaflet valves), for example which can be opened by a rod or other member such as the syringe bar described above. Preferred valves are configured to open automatically either when the Luer assembly is engaged with the valve assembly, or when all three of the syringe, Luer assembly, and valve assembly are connected. Also, as indicated above, other types of valves and connectors or Leur connectors can be employed on the proximal end of the balloon catheter in other embodiments.

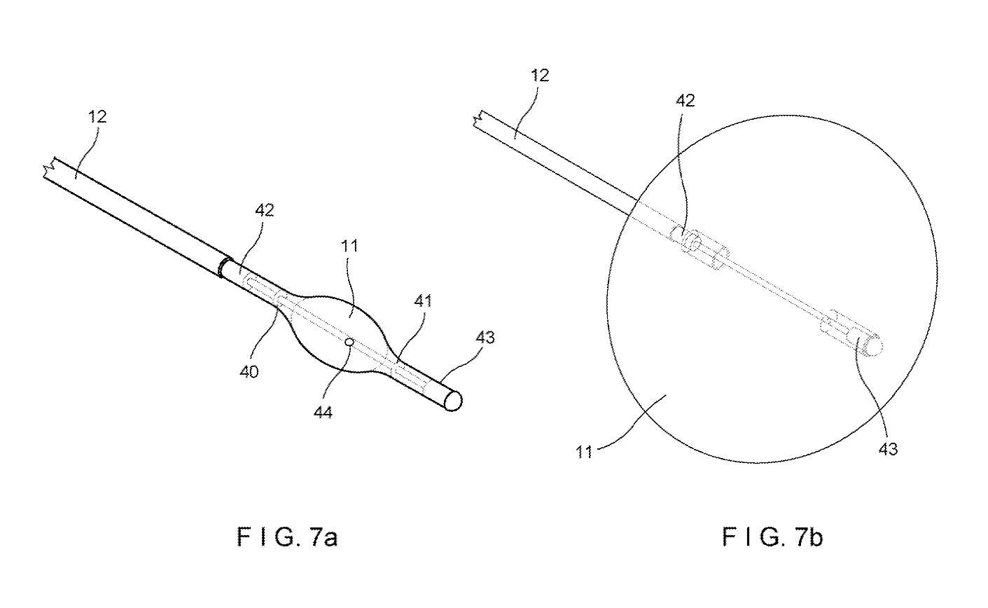

With reference to FIGS. 7a and 7b, balloon 11 is preferably made of compliant material, for example, a silicon-based compound or other elastic elastomer that can be inflated from relatively small size to relatively large size, so that it may apply pressure to a target area. Balloon 11 may have different shapes, for example, ball shape or elliptical shapes, so when inflated the balloon will effectively engage the luminal space in order to provide the intended balloon's functions as disclosed herein.

Balloon 11, in its non-compressed configuration, for example when outside of a working channel of an endoscope, can have the diameter larger than the diameter of a working channel 6 of an endoscope to facilitate the balloon's expansion to very large diameters. To illustrate, if balloon 11, in a free, non-compressed configuration, has a diameter of about 3 mm and is expanded to a diameter of about 70 mm, the balloon 11, therefore, will expand by approximately 2300% in diameter. In comparison, if balloon 11 in a free, non-compressed configuration, has a diameter of about 10 mm and is expanded to a diameter of a 70 mm it will expand by approximately 700% in diameter, which exerts substantially less stress on the balloon's material and allows additional materials (for example, silicone) to be used for making such balloons. When needed, such balloons can, for example, be folded in order to fit into a working channel of an endoscope.

To reduce the diameter of the catheter 10 at the level of balloon 11 to facilitate the passage of the device into and through a working channel 6 of an endoscope, a smaller shaft assembly 40 can be provided. Shaft assembly 40 preferably includes small tube 41 in the distal end, which is preferably made from a relatively stiff material (for example, nitinol tube or polymer-reinforced tube). Tube 41 of the embodiment shown is connected to two larger tubes 42 at its proximal end, and 43 at its distal end. Tubes 42,43 have diameters that fit into an internal diameter of the shaft 12 and balloon 11 and their necks, with tube 42 fitted to the shaft 12 at its distal side 13. Tube 41 preferably includes at least one opening 44 for passage of inflation fluid to the balloon 11, and tubes 41-43 are preferably fitted within the balloon, which preferably holds the ends of tubes 41-43.

The presence of balloon 11 inside a lumen or organ 1 can be used to guide a laparoscopic surgeon (operating in the abdominal cavity, and using the balloon as a marker of the lesion's location), and, hence, allow decreasing operating time, simplify, and minimize the operation. The balloon catheter 10 of the invention can be used for endoscopic procedures in other body lumens, such as in the small bowel, vessel, heart, bronchus, biliary duct, stomach, esophagus, and duodenum.

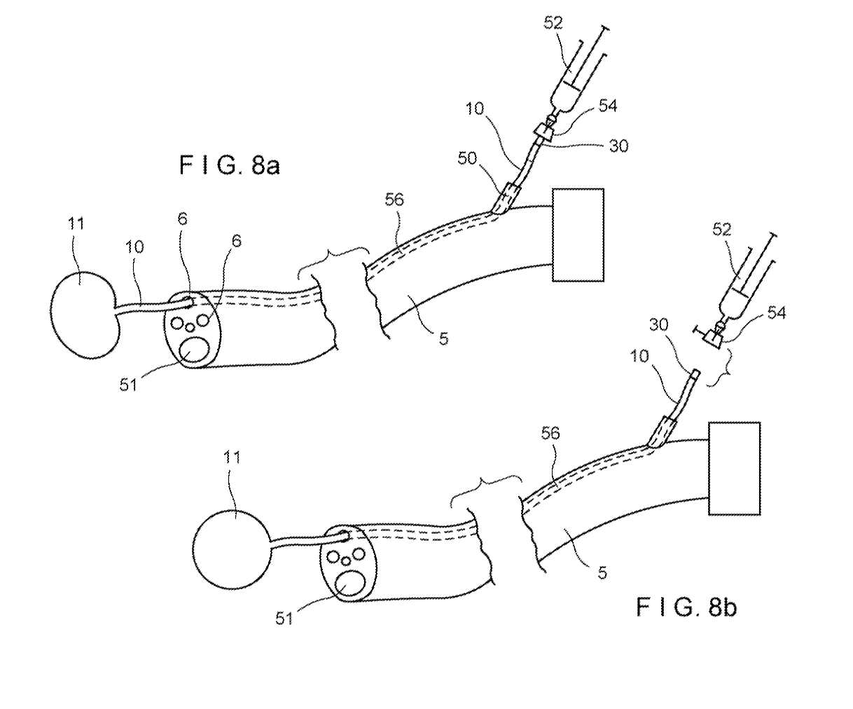

FIG. 8a shows the balloon catheter 10 received in endoscope 5, which is inserted within a patient, and has working openings and provisions for a camera 51 or fiber optics therefor. The catheter's balloon 11 is inflated with fluid from syringe 52. A proximal side of the catheter 10 protrudes from an endoscope port 50 at a proximal end thereof, and distal side from a working opening 6. Syringe 52 is connected to a Luer connector member 54, which is associated with a valve assembly 30, so that the valve is open and in the syringe 52 is in fluid communication with the balloon 11. In FIG. 8b, the syringe 52 and connector member 54 have been detached from the valve assembly 30 so that the valve is automatically or manually closed, and the balloon 11 remains inflated. Since the valve assembly 30 preferably fits through the working opening 6 through the entire endoscopic working channel 56, or shaft, leading between the port 50 and the opening 6, the endoscope 10 can be removed from the patient, with the valve assembly 30 sliding into the port 50 and out of the working opening 6, without deflating the balloon 11. The endoscope 10 can then be reinserted into the patient, sliding the valve assembly 30 into the working opening and out the port 50.

An example of a suitable device that can be used with the present disclosure is described in U.S. Patent Application No. 61/287,077, the entire disclosure of which is hereby incorporated by reference thereto.

As used herein, the terms “top,” “bottom,” and/or other terms indicative of direction are used herein for convenience and to depict relational positions and/or directions between the parts of the embodiments. It will be appreciated that certain embodiments, or portions thereof, can also be oriented in other positions. In addition, the term “about” or “approximately” should generally be understood to refer to both the corresponding number and a range of numbers. In addition, all numerical ranges herein should be understood to include each whole integer within the range.

While illustrative embodiments of the invention are disclosed herein, it will be appreciated that numerous modifications and other embodiments may be devised by those skilled in the art. For example, the features for the various embodiments can be used in other embodiments. Therefore, it will be understood that the appended claims are intended to cover all such modifications and embodiments that come within the spirit and scope of the present invention.

What is claimed is:

1. A catheter assembly, comprising:
a catheter shaft defining an interior channel and having an outer cross-sectional size selected to be received through a working opening of an endoscope; and
a valve associated with the catheter shaft in fluid connection with the interior channel for opening and closing the interior channel, the valve being configured for fluid connection to a connector member for passing a fluid between the connector member and the interior channel when the valve is open, wherein the valve has an outer cross-section configured and dimensioned for reception through the working opening when detached from the connector member.

2. The catheter assembly, wherein the catheter is a balloon catheter that includes a balloon in fluid connection with the interior channel for inflation and deflation by passing the fluid through the channel.

3. The catheter assembly of claim 2, wherein the catheter is a gastrointestinal balloon catheter and the endoscope is a colonoscope.

4. The catheter assembly of claim 1, further comprising the connector member, which includes a Luer connector.

5. The catheter assembly of claim 1, further comprising the connector member, wherein the valve and connector member are configured to cause the valve to open automatically based on a connection between the valve and connector member, and to cause the valve to close automatically upon disconnection thereof.

6. The catheter assembly of claim 5, wherein the connector member includes a valve opening member configured to displace a portion of the valve to open the valve.

7. The catheter assembly of claim 6, wherein the valve comprises a valving obstruction having a closed position blocking flow through the valve to close the valve, and the valve open member is configured for engaging and moving the valving obstruction to an open position unblocking the flow through the valve to open the valve when the connection member and valve are connected.

8. The catheter assembly of claim 7, wherein the connector member defines an interior connector channel, and the valve opening member is disposed within the connector channel and has a position extending from the connector channel into the valve in association with the obstruction for moving the obstruction to the open position.

9. The catheter assembly of claim 8, further comprising a biasing member resiliently biasing the valving obstruction towards the closed position.

10. The catheter assembly of claim 8, wherein the valve opening member is disposed in the connector channel such that connecting the connector member to a syringe causes the syringe to displace the valve opening member against the valving member for opening the valve.

11. The catheter assembly of claim 5, wherein:
the valve comprises a seal; and
the connector member defines an interior connector channel and includes a piercing member connected with the connector channel and configured to pierce the seal to open fluid communication between the connector channel and the interior channel of the shaft when the connector member and valve are connected.

12. The catheter assembly of claim 1, wherein:
the shaft has an outer shaft diameter at a distal end portion thereof; and
the valve is connected to the distal end portion and has an outer valve diameter substantially the same or smaller than the shaft diameter.

13. The catheter assembly of claim 12, wherein the valve diameter is no larger than the shaft diameter.

14. The catheter assembly of claim 12, wherein the valve diameter is smaller than the shaft diameter.

15. The catheter assembly of claim 1, wherein the connector member comprises a fitting configured for engaging the valve in a releasable snap connection therewith.

16. An endoluminal treatment method, comprising:
providing a balloon catheter connected to a connector member;
inflating a balloon of the balloon catheter, which balloon catheter is received through a working opening of an endoscope and is positioned endoluminally;
detaching the inflated balloon catheter from a connector member, thereby closing a valve associated with the catheter to block fluid flow from the catheter; and
removing the endoscope from the catheter by sliding the valve through the endoscope's working opening.

17. The method of claim 16, wherein the balloon is inflated to apply pressure against a bleeding site to stop or slow the bleeding.

18. The method of claim 16, wherein the balloon is inflated and positioned to block a perforated region in a wall of the lumen.

19. The method of claim 16, wherein the balloon catheter is a gastrointestinal balloon catheter, and the endoscope is a colonoscope.

20. The method of claim 16, wherein:
the connector member is a Luer connector;
the balloon catheter is inflated using a syringe connected to the Luer connector; and
the method further comprises connecting a syringe to the Luer connector after removal of the endoscope for deflating the balloon.
